(19) United States
(12) Patent Application Publication
(10) Pub. No.: US 2020/0297092 A1
(43) Pub. Date: Sep. 24, 2020

(54) Hair Iron Sheath

(71) Applicants: Veronica Green, Tilbury (CA); Michael J. Green, Tilbury (CA)

(72) Inventors: Veronica Green, Tilbury (CA); Michael J. Green, Tilbury (CA)

(21) Appl. No.: 16/357,218

(22) Filed: Mar. 18, 2019

(51) Int. Cl. A45D 6/00; A45D 1/04; A45D 7/02; A61K 8/92; A61Q 5/12

(52) U.S. Cl. CPC A45D 6/00; A45D 1/04; A45D 2200/155; A61K 8/92; A61Q 5/12; A45D 7/02

(57) ABSTRACT

A hair iron having a first arm having a first free end, a first joint end, a first heating element and a first hair heating plate on an interior surface thereof proximate the first free end. The first heating element providing heat to the first hair heating plate. A second arm having a second free end, a second joint end, a second heating element and a second hair heating plate on an interior surface thereof proximate the second free end. The second heating element providing heat to the second hair heating plate. The first and second arms joined at the first and second join ends via a joint member such that the arms can be flexibly opened and closed to bring the first and second hair heating plates into and out of contact. A first removable, heat-resistant hair iron sheath, the first hair iron sheath configured to wrap around the first hair iron arm covering the first hair heating plate. A second removable, heat-resistant hair iron sheath, the second hair iron sheath configured to wrap around the second hair iron arm covering the second hair heating plate.

### REFERENCE TO PENDING APPLICATIONS

This application does not claim the benefit of any issued U.S. Patent or pending application.

### TECHNICAL FIELD

The present invention generally relates to a hair iron for hairstyling, and more specifically, to a grooming sheath that covers the heating plate of a hair iron.

### BACKGROUND

Hairstyling has always been an important factor for a decent or attractive appearance and when your hair looks good, you look good and it brings out their self confidence. Today, hair irons are conveniently and widely used not only by hairstyling professionals but also by women at home as a tool for simple hair setting or hairstyling.

There are different types of hair, i.e., straight hair, frizzy hair, curly hair, and the like. In general, in order to attain a desired hairstyle, for example, straight hair is set with curls or curly hair is straightened. Easy hairstyling using hair irons is now possible and thus, a demand for hair iron products has steadily increased as a reflection of consumers' aesthetic tastes.

While the use of hair irons has become wide spread, hair damage caused by drastic hairstyling and erroneous home haircare such as excessive hair washings has become a serious problem. Thus, there is a strong demand for development of hair irons that can reduce or prevent damage to the hair.

Accordingly, there is a need for an apparatus and method to address the issues set out above.

### SUMMARY

The present invention generally relates to a hair iron for hairstyling, and more specifically, to a grooming sheath that covers the heating plate of a hair iron.

An aspect of the hair iron of the present invention is disclosed. This aspect includes a first arm having a first free end, a first joint end, a first heating element and a first hair heating plate on an interior surface thereof proximate the first free end. The first heating element provides heat to the first hair heating plate. This aspect further includes a second arm having a second free end, a second joint end, a second heating element and a second hair heating plate on an interior surface thereof proximate the second free end. The second heating element provides heat to the second hair heating plate. The first and second arms are joined at the first and second join ends via a joint member such that the arms can be flexibly opened and closed to bring the first and second hair heating plates into and out of contact.

This aspect further includes a first and a second removable, heat-resistant hair iron sheath, or sheath. Each cover is configured to wrap around one of the arms covering the heating plate thereon. Each cover may be constructed from a sheet of paper having a conditioning coating, such as wax. The coating, when heated, transfers to the user' hair to provide the desired hairstyling effects while reducing damage to the hair. The cover may also be reusable or may be configured for a single use.

Other aspects and features of the present invention will become apparent to those ordinarily skilled in the art upon review of the following description of specific embodiments of the invention in conjunction with the accompanying figures.

### BRIEF DESCRIPTION OF THE DRAWING

In drawings which illustrate embodiments of the invention wherein similar characters of reference denote corresponding parts in each view,

FIG. 1 is a perspective view of a prior art hair iron.

FIG. 2 is a perspective view of an embodiment of a hair iron of the present invention.

FIG. 3 is a cross-section view of an embodiment of a hair iron sheath of the present invention.

FIG. 4 is a perspective view of an embodiment of a hair iron sheath of the present invention.

### DETAILED DESCRIPTION OF THE INVENTION

The present invention is directed toward a grooming sheath that is placed over an arm of a hair iron. The sheath is a covering that includes a body being closed at one end and open at a second end, and defining an interior. The open end is designed to allow an arm of a hair iron to enter into the interior. The sheath slides over the arm covering both the heating plate, or front, side and the back side of the arm. This allows for the continuity of the sheath during use. For example, the sheath covered hair iron will be able to glide through the hair smoothly sealing in the hair curl as it is curled around the hair iron.

In some embodiments, the open sheath may be secured to the arm by a fastener, such as an elastic band sewn on the inside of the sheath around an inch or so past the heating plate elements but short enough as to not cover any buttons that are on the inside of some older heat flat irons.

The present invention prevents hair damage. When using a hair iron, due to the heat of the hair iron, the hair iron must be moved very quickly through the hair otherwise the hair can be damaged from the heat of the hot plates. The present invention allows for the transfer of heat from the hair iron without the pressure of working either too fast or too slow.

The present invention will allow for the protection of the hair iron by preventing any build up on the hot plates that is caused by dead hair cells, product build up, etc., which in turn can be transferred to the user's hair.

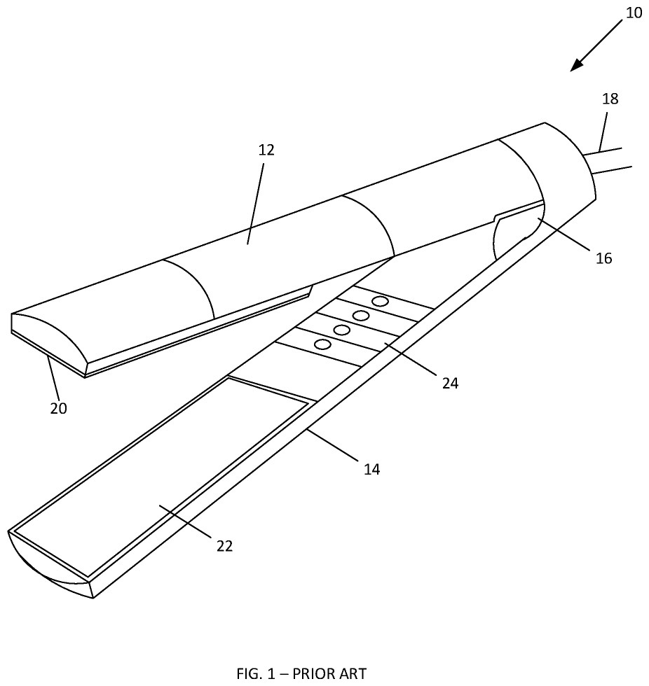

A general prior art arm-type hair iron is shown in FIG. 1, in which the hair iron 10 includes two arms 12 and 14 which are joined by a joint 16. Each arm has a heating plate 20, 22, and a cord 18 connectable to a source of electrical power. In this iron, the arms can be freely opened and closed by the gripping force of a hand, and the hair can be ironed by sandwiching a bundle of hair between the arms and pressing the heating plates together with the hand. A heating element, not shown is located within each arm and provides heat to each heating plate 20, 22. A set of control buttons 24 are typically included on at least one of the arms.

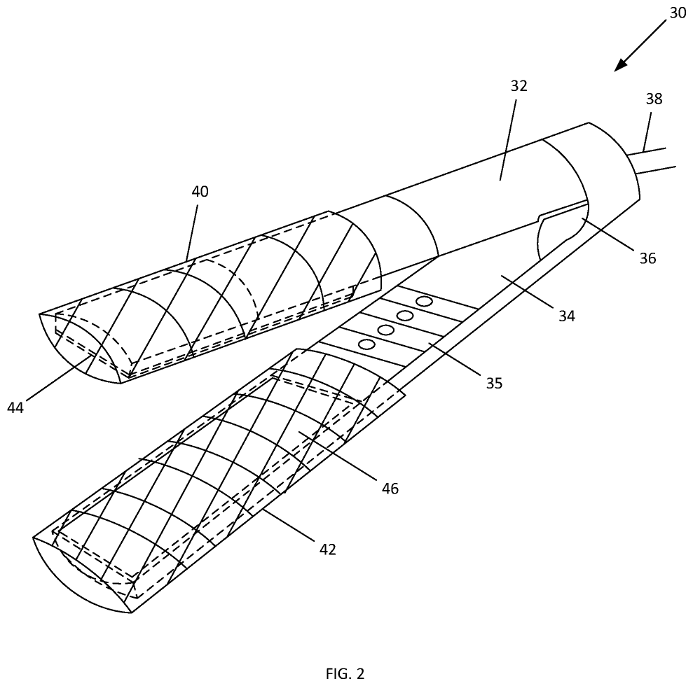

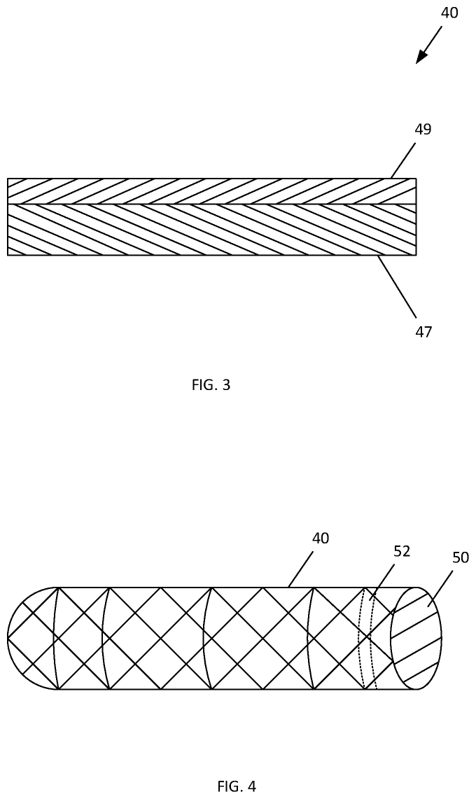

As illustrated in FIGS. 2-4, an embodiment of a hair iron 30 of the present invention is disclosed. As with the above discussed prior art arm-type hair iron, hair iron 30 includes two arms 32 and 34 which are joined by a joint 36. Each arm has a heating plate 44, 46, and a cord 38 connectable to a source of electrical power. A heating element, not shown is located within each arm 32, 34 and provides heat to each heating plate 44, 46.

The free ends of arm 32, 34 are inserted into hair iron sheaths 40, 42 through an opening 50 therein. The covers 40, 42 configured to wrap around the arms 32, 34 and covering the heating plates 44, 46. Further, covers 40, 42 may be secured to the arms 32, 34 by way of an elastic band 62, see FIG. 4, or similar type of securing fastener. Covers 40, 42 are removable and heat-resistant. In some embodiments, covers 40, 42 are constructed from a sheet of paper 47 having a wax coating 49. The paper sheeting being configured to wrap around the at least one hair iron arm covering the heating plate. The covers 40, 42 may be configured to extend only to cover the heating plates 44, 46 or may extend down along the length of the arms 32, 34.

The wax may be any type of wax or paraffin which is configured to condition the user's hair upon being heated and applied to the such hair. The use of wax is illustrative. Those skilled in the art will recognize that other types of hair conditioners, such as Keratin micro-conditioners, may be utilized and are within the scope of the present invention.

As shown in FIG. 4, the cover 40 may be is formed to be cylindrical to wrap around the arms 32, 34 of the iron 30 so that the heat of the heating plates 44, 46 may reach the entire periphery of the hair through the cover, which increases the heating efficiency of the heating surface during the hair treatment.

While preferred embodiments of the present inventive concept have been shown and disclosed herein, it will be obvious to those persons skilled in the art that such embodiments are presented by way of example only, and not as a limitation to the scope of the inventive concept. Variations, changes, and substitutions may occur or be suggested to those skilled in the art without departing from the intent, scope, and totality of this inventive concept. Such variations, changes, and substitutions may involve other features which are already known per se and which may be used instead of, in combination with, or in addition to features already disclosed herein. Accordingly, it is intended that this inventive concept be inclusive of such variations, changes, and substitutions, and by no means limited by the scope of the claims presented herein.

I claim:

1. A hair iron, comprising:
a first arm having a first free end, a first joint end, a first heating element and a first hair heating plate on an interior surface thereof proximate the first free end, the first heating element providing heat to the first hair heating plate;
a second arm having a second free end, a second joint end, a second heating element and a second hair heating plate on an interior surface thereof proximate the second free end, the second heating element providing heat to the second hair heating plate, the first and second arms joined at the first and second join ends via a joint member such that the arms can be flexibly opened and closed to bring the first and second hair heating plates into and out of contact;
a first removable, heat-resistant hair iron sheath, the first hair iron sheath configured to wrap around the first hair iron arm covering the first hair heating plate; and
a second removable, heat-resistant hair iron sheath, the second hair iron sheath configured to wrap around the second hair iron arm covering the second hair heating plate.

2. The hair iron of claim 1, wherein the press covers are comprised of a sheet of paper having a hair conditioner coating, the paper sheeting being configured to wrap around the at least one hair iron arm covering the heating plate.

3. The hair iron of claim 1, wherein the hair conditioner coating is a wax coating.

4. The hair iron of claim 1, wherein the press covers completely cover the first and second free ends of the first and second arms.

5. A hair iron sheath for use with a hair iron having at least one hair iron arm, the at least one hair iron arm having a heating plate on an interior surface thereof, the hair iron sheath comprising:
a sheet of paper having a hair conditioner coating, the paper sheeting being configured to wrap around the at least one hair iron arm covering the heating plate.

6. The hair iron sheath of claim 5, wherein the hair conditioner coating is a wax coating.
